(19) United States
(12) Patent Application Publication
(10) Pub. No.: US 2022/0180771 A1
(43) Pub. Date: Jun. 9, 2022

(54) System for experience educating vital signs and cardiopulmonary

(71) Applicant: New EM Co., Ltd., Chuncheon-si (KR)

(72) Inventor: Seong Min KANG, Chuncheon-si (KR)

(21) Appl. No.: 17/120,625

(22) Filed: Dec. 14, 2020

(30) Foreign Application Priority Data
Dec. 3, 2020 (KR) 10-2020-0167500

(51) Int. Cl. G09B 23/30; A61B 5/00; A61B 5/02

(52) U.S. Cl. CPC G09B 23/30; A61B 5/02; A61B 5/742

(57) ABSTRACT

A system for experience educating vital signals and cardiopulmonary according to the present disclosure may include: a vital signal measurement unit having a vital signal measurement probe; a human body model formed in the shape of a human body; and a processor setting a vital signal output mode on the basis of the vital signal recognition information when the vital signal measurement data and the vital signal recognition information are input, checking output forma information corresponding to the vital signal output mode, and controlling an output unit and a display unit respectively to output and display the vital signal measurement data in correspondence to the output format information.

### CROSS-REFERENCE TO RELATED APPLICATIONS

This application claims the benefit under 35 U.S.C. section 119, which claims foreign priority to Korean Patent Application No. KR10-2020-0167500, filed on Dec. 3, 2020, in the Korean Intellectual Property Office, the disclosure of which is hereby incorporated by reference its entirety.

### BACKGROUND OF THE DISCLOSURE

### Field of the disclosure

The present disclosure relates to a system for experience educating vital signals and cardiopulmonary and, more particularly, to a system for experience educating vital signals and cardiopulmonary, the system being able to perform experience education for vital signals and cardiopulmonary.

### Related Art

Medical engineering that strives for development of two fields of study by exchanging and applying academic methodology, concept, technology, device, etc. between the fields of medical science and engineering is referred to as various names such as ‘medical & biological engineering’, ‘biomedical engineering’, ‘medical engineering’, and ‘biomedical electronic engineering’, and is very closely connected with medical equipment employing various electronic/communication technologies requiring high precision and reliability.

On the basis of the electronic/communication technologies that are applied, the main field of study of medical engineering can be classified into vital signal process, medical image process and analysis, medical equipment, modeling and simulation, biomechanics, biomaterial, an assistive technology, an artificial organ, medical information, a diagnosis assistant system, etc.

New demand and the market of various items of educational equipment related to electronic/communication technologies that are used in the field of medical engineering are increased with the development of the electronic communication technology related to medical science. However, although the electronic communication technology is being recently rapidly developed such that the corresponding field of education cannot catch up with the development, the educational equipment in the field is substantially very old and it is required to replace educational equipment with the newest products in accordance with various fields of electronic/communication technology.

However, since a vital signal measurement sensor that is the target of education and a circuit for processing signals are integrated in educational experience systems of the related art, education of various vital signal measurement fields is impossible and it is required to purchase a new experience system every time in accordance with changes of the education target and process, which causes a burden of cost.

Further, since the systems are configured in an integrated type, when a problem is generated, assembling and disassembling are difficult and it is complicated and takes long time to repair the systems, and it is very difficult to upgrade various circuits and change the specifications.

### SUMMARY OF THE DISCLOSURE

The present disclosure relates to a system for experience educating vital signals and cardiopulmonary, and more detail, provides a system for experience educating vital signals and cardiopulmonary, the system converting vital signals of a learner measured by a vital signal measurement probe disposed in a vital signal measurement unit into vital signal measurement data to be output by an output unit or displayed by a display unit.

Further, the present disclosure provides a system for experience educating vital signals and cardiopulmonary, the system enabling an output unit or a display unit to output or display vital signal education data when a vital signal measurement probe of a vital signal measurement unit is attached to a human body model.

The objectives of the present disclosure are not limited to those described above and other objectives and advantages not stated herein may be understood through the following description and may be clear by embodiments of the present disclosure. Further, it would be easily known that the objectives and advantages of the present disclosure may be achieved by the configurations described in claims and combinations thereof

In order the achieve the objectives, a system for experience educating vital signals and cardiopulmonary according to the present disclosure may include: a vital signal measurement unit having a vital signal measurement probe that is attached to a learner or a human body model, measuring a vital signal from the learner through the vital signal measurement probe, converting the measured vital signal into vital signal measurement data, and outputting the vital signal measurement data together with vital signal recognition information; a human body model formed in the shape of a human body, and outputting a probe attachment sensing signal together with vital signal identification information corresponding to a vital signal measurement region when the vital signal measurement probe is attached to the vital signal measurement region; and a processor setting a vital signal output mode on the basis of the vital signal recognition information when the vital signal measurement data and the vital signal recognition information are input, checking output forma information corresponding to the vital signal output mode, and controlling an output unit and a display unit respectively to output and display the vital signal measurement data in correspondence to the output format information.

The processor may set the vital signal output mode on the basis of the vital signal recognition information when the probe attachment sensing signal and the vital signal recognition information are input, check output forma information corresponding to the vital signal output mode, and control the output unit and the display unit respectively to output and display vital signal education data in correspondence to the output format information.

The system for experience educating vital signals and cardiopulmonary according to the present disclosure may further include a heart model formed in the shape of the heart of a human body, disposed in the human body model, and expanding and contracting to represent relaxation and contraction of the heart.

When the vital signal measurement data and the vital signal recognition information are input, the processor may control expansion and contraction of the heart model on the basis of the vital signal measurement data.

When the probe attachment sensing signal and the vital signal recognition information are input, the processor may control expansion and contraction of the heart model on the basis of vital signal education data.

The system for experience educating vital signals and cardiopulmonary according to the present disclosure may further include: a display unit displaying the vital signal data or vital signal education data in one or more of a text, an image, and a video; and an output unit printing out the vital signal data or the vital signal education data

According to the present disclosure, a vital signal of a learner measured through a vital signal measurement probe of a vital signal measurement unit is converted into vital signal measurement data and then output or displayed through an output unit or a display unit, whereby the learner can experience measurement of his/her vital signals.

Further, according to the present disclosure, an output unit or a display unit outputs or displays vital signal education data when a vital signal measurement probe of a vital signal measurement unit is attached to a human body model, whereby it is possible to effectively teach a vital signal measurement method.

### BRIEF DESCRIPTION OF THE DRAWINGS

FIG. 1 is a perspective view of a system for experience educating vital signals and cardiopulmonary according to the present disclosure.

FIG. 2 is a view showing a connection configuration of a system for experience educating vital signals and cardiopulmonary according to the present disclosure and a cardiopulmonary resuscitation education assistant device.

FIG. 3 is a view for explaining an example of a function block diagram for explaining a system for experience educating vital signals and cardiopulmonary according to the present disclosure.

FIG. 4 is a front view of a system for experience educating vital signals and cardiopulmonary according to the present disclosure.

FIG. 5 is a view showing the state in which an electrocardiogram vital signal measurement probe of vital signal measurement probes is attached to a human body model of a system for experience educating vital signals and cardiopulmonary.

FIG. 6 is a view showing the state in which an oxygen saturation vital signal measurement probe of vital signal measurement probes is attached to a human body model of a system for experience educating vital signals and cardiopulmonary.

FIG. 7 is a view showing the state in which a blood pressure vital signal measurement probe of vital signal measurement probes is attached to a human body model of a system for experience educating vital signals and cardiopulmonary.

FIG. 8 is a view showing the state in which a system for experience educating vital signals and cardiopulmonary according to the present disclosure and a cardiopulmonary resuscitation education assistant device are connected.

### DESCRIPTION OF EXEMPLARY EMBODIMENTS

Hereafter, various embodiments of the present disclosure are described with reference to accompanying drawings. However, this is not intended to limit the present disclosure to specific embodiments and should be construed as including various modifications, equivalents, and/or alternatives of the embodiments of the present disclosure. In the description of drawings, similar components may be given similar reference numerals.

In the specification, expressions such as “have”, “may have”, “include”, or “may include” indicate existence of corresponding characteristics (e.g., a number, a function, an operation, or a component such as a part) without excluding existence of additional characteristics.

In the specification, the terms “A or B”, “at least one of A and/or B”, or “one or more of A, B and/or C” may include all possible combinations of items to be enumerated. For example, “A or B”, “at least one of A and B”, or “at least one of A or B” may indicate all of a case (1) including at least one A, a case (2) including at least one B, or a case (3) including both of at least one A and at least one B.

The terms such as “first” and “second” used in various embodiments may modify various components regardless of the order and/or priority and are used only to discriminate one component from another component without limiting the components. For example, a first user device and a second user device may refer to different user devices regardless of the sequence or priority. For example, the first component may be named the second component, and vice versa, without departing from the scope of the present disclosure.

When a component (e.g., a first component) is (“operatively or communicatively) coupled with/to or connected to another component (e.g., a second component), it should be understood that the component may be connected to the another component directly or through another component (e.g., a third component). However, when a component (e.g., a first component) is “directly coupled to” or “directly connected” to another component (e.g., a second component), it may be understood as another component (e.g., a third component) may exist between the component and the another component.

The terms used herein “configured to” may be replaced, for example, with “suitable for”, “having the capacity to”, “designed to”, “adapted to”, “made to”, or “capable of”, depending on circumstances. The term “configured to” may not refer only “specifically designed to” in term of hardware. Instead, in some circumferences, the term “device configured to” may refer to that the device “is capable of doing” with other devices or parts. For example, a “controller configured to perform expressions A, B, and C” may refer to an exclusive processor (e.g., an embedded processor) for performing the corresponding operations or a generic-purpose processor (e.g., a CPU or an application processor) being capable of performing the corresponding operations by executing one or more software programs stored in a memory.

Further, the term “˜unit” used herein means a software component or a hardware component such FPGA, or ASIC and performs predetermined functions. However, the term “˜unit” is not limited to software or hardware. The term “˜unit” may be configured to be in a storage medium that can be addressed. Accordingly, for example, the “unit” includes components such as software components, object-oriented software components, class components, and task components, processors, functions, properties, procedures, subroutines, segments of a program code, drivers, firmware, a microcode, a circuit, data, a database, data structures, tables, arrays, and variables. Functions provided by the components and the “units” may be combined in a smaller number of components and “units” or may be further separated into additional components and “units”.

In particular, in this specification, a “processor” may include one or more of a Central Processing Unit (CPU), an Application Processor (AP), and a Communication Processor (CP).

In this specification, a computer means all kinds of hardware device including at least one processor, and depending on embodiments, may be understood as a meaning including even a software configuration that is operated in a corresponding hardware device. For example, a computer may be understood as a meaning that includes all of a smartphone, a tablet PC, desktop, a notebook, and user clients and applications that are driven in each device, but is not limited thereto.

Terminologies used herein may be used only to describe specific embodiments without intention of limiting the range of other embodiments. Singular forms are intended to include plural forms unless the context clearly indicates otherwise. All terminologies used herein including technological or scientific terminologies may have the same meanings that are generally understood by those skilled in the art. Terminologies defined in general dictionaries of the terminologies used herein may be understood as having meanings the same as or similar to the meanings in the contexts and should not be construed as abnormally or exclusively formally meanings unless specifically defined herein. Depending on cases, even if terminologies defined herein, they should not be construed as excluding the embodiments of the present disclosure.

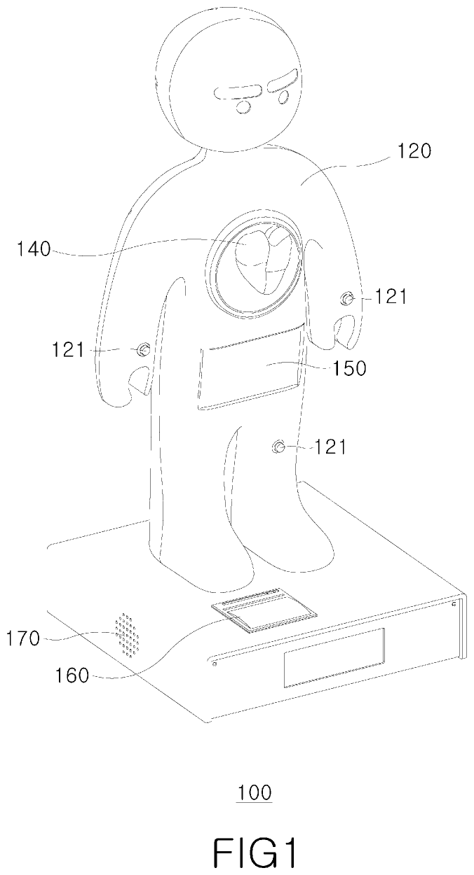

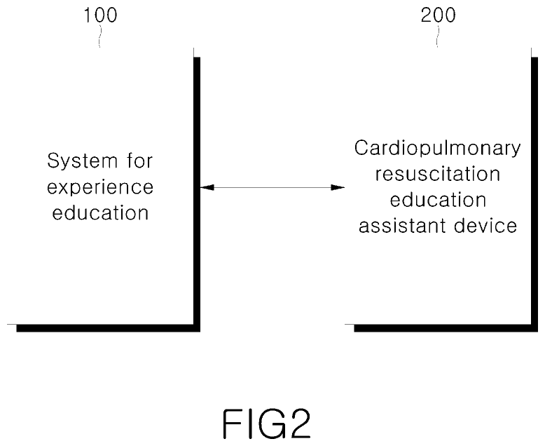

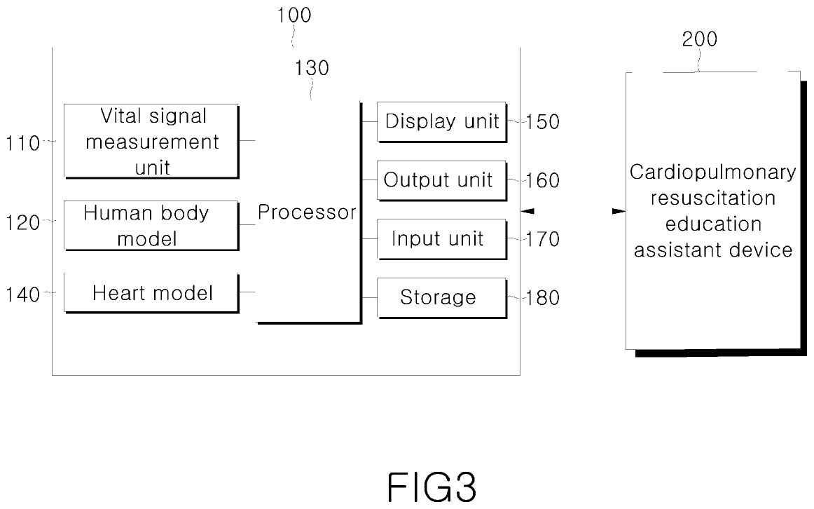

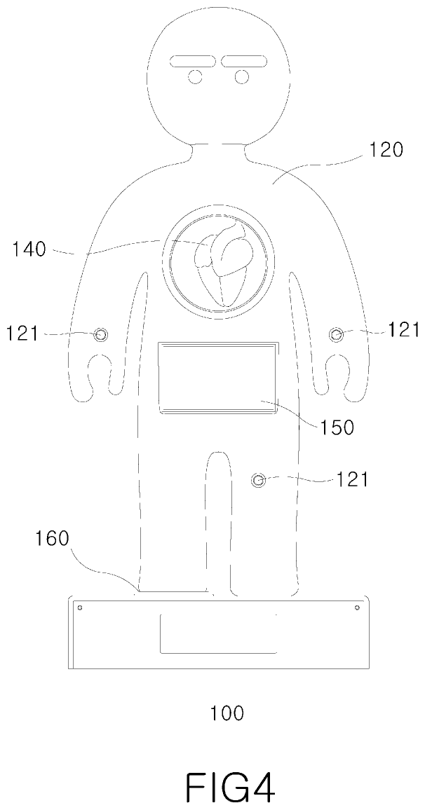

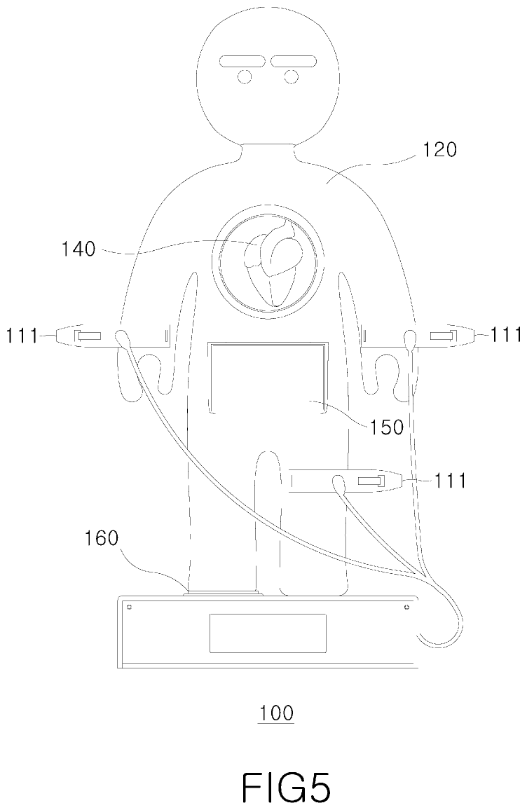

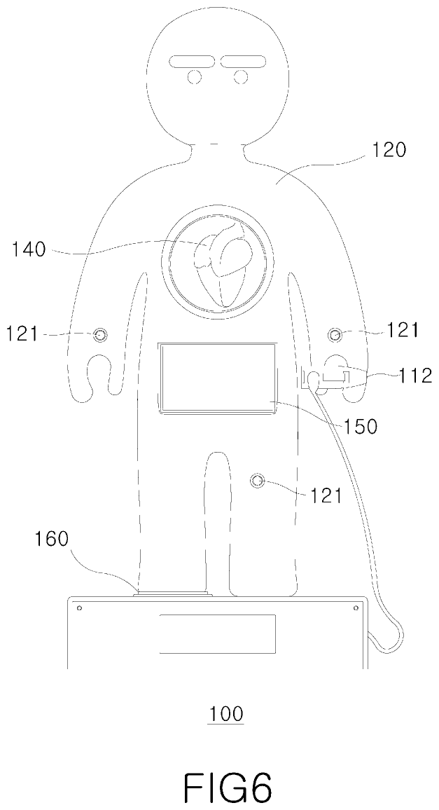

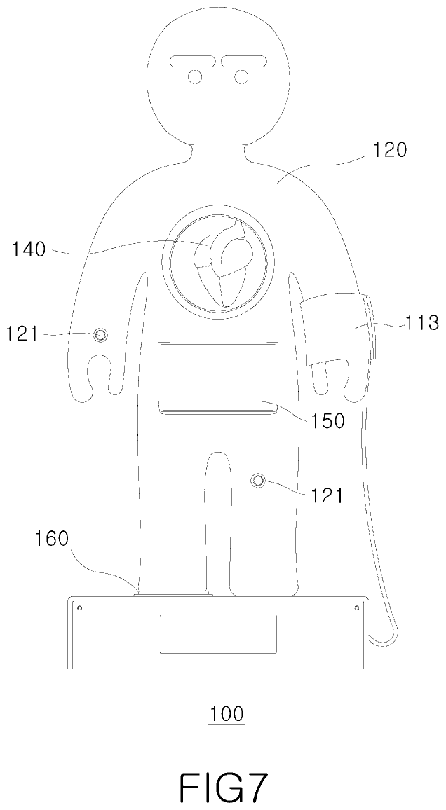

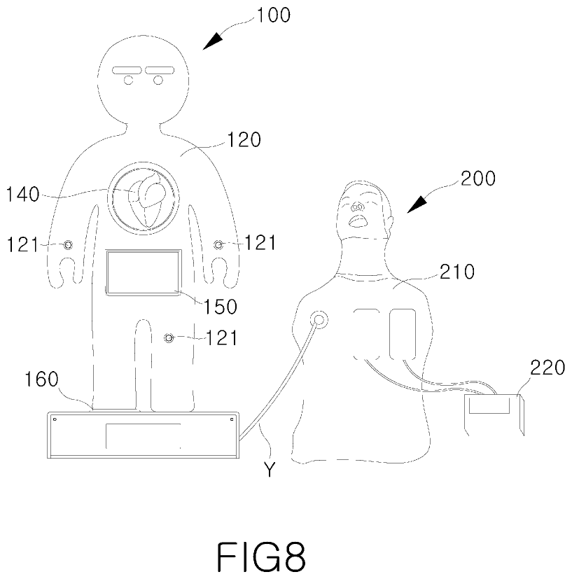

FIG. 1 is a perspective view of a system 1000 for experience educating vital signals and cardiopulmonary according to the present disclosure, FIG. 2 is a view showing a connection configuration of the system 1000 for experience educating vital signals and cardiopulmonary according to the present disclosure and a cardiopulmonary resuscitation education assistant device 200, FIG. 3 is a view for explaining an example of a function block diagram for explaining the system 1000 for experience educating vital signals and cardiopulmonary according to the present disclosure, FIG. 4 is a front view of the system 1000 for experience educating vital signals and cardiopulmonary according to the present disclosure, FIG. 5 is a view showing the state in which an electrocardiogram vital signal measurement probe 111 of vital signal measurement probes is attached to a human body model of the system 1000 for experience educating vital signals and cardiopulmonary, FIG. 6 is a view showing the state in which an oxygen saturation vital signal measurement probe 112 of vital signal measurement probes is attached to a human body model of the system 1000 for experience educating vital signals and cardiopulmonary, FIG. 7 is a view showing the state in which a blood pressure vital signal measurement probe 113 of vital signal measurement probes is attached to a human body model of the system 1000 for experience educating vital signals and cardiopulmonary, and FIG. 8 is a view showing the state in which the system 1000 for experience educating vital signals and cardiopulmonary according to the present disclosure and a cardiopulmonary resuscitation education assistant device 200 are connected.

Referring to FIG. 1, a system 100 for experience educating vital signals and cardiopulmonary may include a vital signal measurement unit 110, a human body model 120, a processor 130, a heart model 140, a display unit 150, an output unit 160, an input unit 170, and a storage 180.

The vital signal measurement unit 110 can measure vital signals of a learner.

To this end, the vital signal measurement unit 110 may include vital signal measurement probes 111, 112, and 113 that are attached to a learner.

The vital signal measurement probes may be members that are attached to the body of a learner and measure vital signals of the learner.

For example, as shown in FIG. 5, the vital signal measurement probe may be an electrocardiogram vital signal measurement probe 111 that can measure electrocardiogram vital signals.

The electrocardiogram vital signal measurement probe 111 may have an electrode for measuring an electrocardiogram vital signal and may have a wire transmitting an electrocardiogram vital signal.

Three electrocardiogram vital signal measurement probes 111 may be provided and attached to both hands and legs of a learner.

As another example, as shown in FIG. 6, the vital signal measurement probe may be an oxygen saturation vital signal measurement probe 112 that can measure oxygen saturation vital signals.

The oxygen saturation vital signal measurement probe 112 may have an optical sensor for measuring an oxygen saturation vital signal and may have a wire transmitting an oxygen saturation vital signal.

The oxygen saturation vital signal measurement probe 112 may be attached to the end of a finger of a learner.

As another example, as shown in FIG. 7, the vital signal measurement probe may be a blood pressure vital signal measurement probe 113 that can measure blood pressure vital signals.

The blood pressure vital signal measurement probe 113 may have a pressing module and a pressure sensor for measuring a blood pressure vital signal and may have a wire transmitting a blood pressure vital signal.

The blood pressure vital signal measurement probe 113 may be attached to an arm of a learner.

The vital signal measurement unit 110 can measure vital signals (e.g., an electrocardiogram vital signal, an oxygen saturation vital signal, a blood pressure vital signal) through the vital signal measurement probes 111, 112, and 113, convert the measured vital signals into vital signal measurement data (e.g., electrocardiogram vital signal measurement data, oxygen saturation vital signal measurement data, blood pressure vital signal measurement data), and output the data with vital signal recognition information to the processor 130.

To this end, the vital signal measurement unit 110 may have an electrocardiogram sensor measuring an electrocardiogram vital signal, an oxygen saturation sensor measuring an oxygen saturation vital signal, and a blood pressure sensor measuring a blood pressure vital signal.

It should be noted that the kind and measurement type of the sensors measuring vital signals are not limited.

That is, the vital signal measurement unit 110 can measure any one of an electrocardiogram vital signal, an oxygen saturation vital signal, and a blood pressure vital signal as a vital signal.

Thereafter, the vital signal measurement unit 110 can create vital signal measurement data that are digital data by converting the vital signals that are analog signals.

In detail, the vital signal measurement unit 110 can convert an electrocardiogram vital signal into electrocardiogram vital signal measurement data when an electrocardiogram vital signal is measured, can convert an oxygen saturation vital signal into oxygen saturation vital signal measurement data when an oxygen saturation vital signal is measured, and can convert a blood pressure vital signal into blood pressure vital signal measurement data when a blood pressure signal is measured.

In more detail, the vital signal measurement unit 110 can set a vital signal conversion mode on the basis of vital signal recognition information, can filter and modulate vital signals in correspondence to the vital signal conversion mode, and can create the modulated vital signals as vital signal measurement data.

The vital signal measurement unit 110 checks filtering frequency range information corresponding to the vital signal conversion mode and can filter vital signals, which have a vital signal frequency that is included in the frequency range according to the filtering frequency range information, from vital signals. To this end, the vital signal measurement unit 110 may include a high-pass filter and a low-pass filter.

The vital signal conversion mode may be set to be different for the kind of each vital signal. That is, the vital signal conversion module may mean a mode in which filtering frequency range information is set for the kind of each vital signal according to the vital signal recognition information.

The vital signal measurement unit 110 checks amplitude modulation information corresponding to the vital signal conversion mode and can modulate the vital signal amplitude of a vital signal using the amplitude magnification according to the amplitude modulation information. To this end, the vital signal measurement unit 110 may include a signal amplifier.

The vital signal conversion module may mean a mode in which amplitude modulation information showing a different amplitude magnification is set for the kind of each vital signal according to the vital signal recognition information.

Meanwhile, the vital signal measurement unit 110 can filter out a specific frequency of a vital signal that is an analog signal consisting of a voltage signal or a current signal, using the method described above, and can modulate the amplitudes of vital signals not filtered.

Thereafter, the vital signal measurement unit 110 can convert the filtered and modulated vital signals into vital signal measurement data that are digital signals. To this end, the vital signal measurement unit 110 may include an ADC circuit.

Finally, the vital signal measurement unit 110 can output vital signal recognition information to the processor 130 together with the vital signal measurement data.

When vital signal measurement data and vital signal recognition information are input from the vital signal measurement unit 110, the processor 130 can set a vital signal display mode on the basis of the vital signal recognition information and can control the display unit 150 to display the vital signal measurement data or control the output unit 160 to output vital signal measurement data in correspondence to the vital signal display mode.

The vital signal display mode may be a mode in which output form information for displaying vital signal measurement data for the kind of each signal according to vital signal recognition information has been set.

That is, the processor 130 can control the display unit 150 or the output unit 160 to display or output vital signal measurement data in any one of a slope graph form, a histogram form, and a numeral form according to the output form information.

The display unit 150 can display the vital signal measurement data on a screen using one or more of a text, an image, and a video and the output unit 160 can print out the vital signal measurement data on a paper.

Meanwhile, the heard model 140 is formed in the shape of the heart of human, is disposed in the human body model 120, and can expand and contract to represent relaxation and contraction of the heart.

To this end, the heart model 140 may include a motor therein.

Meanwhile, when vital signal measurement data and vital signal recognition information are input, the processor 130 can control expansion and contraction of the heart model 140 on the basis of the vital signal measurement data.

In detail, the processor 130 can check heart rate information corresponding to the vital signal measurement data and can control expansion and contraction of the heart model 140 at the heart rate according to the check heart rate information.

Meanwhile, the vital signal measurement probes 111, 112, and 113 may be attached to vital signal measurement regions corresponding to a body part, to which the vital signal measurement probes 111, 112, and 113 are attached when a vital signal is measured, of the vital signal measurement regions of the human body model 120.

For example, the electrocardiogram vital signal measurement probe 111 may be attached to a vital signal measurement region corresponding to arms and legs that are body parts, to which the electrocardiogram vital signal measurement probe 111 is attached when an electrocardiogram vital signal is measured, of regions of the human body model 120 by a learner.

The human body model 120 is formed in the shape of a human body, and when the vital signal measurement probe is attached to a vital signal measurement region, the human body model 120 can output a probe attachment sensing signal together with vital signal recognition information corresponding to vital signal measurement region.

For example, the human body model 120 may be divided into a vital signal measurement region corresponding to both forearms and legs that are body parts for measuring an electrocardiogram vital signal, a vital signal measurement region corresponding to the ends of fingers that are body parts for measuring an oxygen saturation vital signal, and a vital signal measurement region corresponding to arms that are body parts for measuring a blood pressure vital signal. The human body model 120 can sense whether a vital signal measurement probe has been attached to each vital signal measurement region. To this end, any one of a button, a pressure sensor, and an illumination sensor may be disposed in each of the vital signal measurement regions of the human body model 120.

For example, the human body model 120 may have a button 121 that senses whether an electrocardiogram vital signal measurement probe 111 is attached to the vital signal measurement region corresponding to both forearms and legs that are body parts for measuring an electrocardiogram vital signal.

Thereafter, when it is sensed that the vital signal measurement probes 111, 112, and 113 are attached to the vital signal measurement regions, the human body model 120 can output a probe attachment sensing signal to the processor 130 together with vital signal recognition information corresponding to a corresponding vital signal measurement region.

When a probe attachment sensing signal and vital signal recognition information are input from the human body model 120, the processor 130 can set a vital signal display mode on the basis of the vital signal recognition information and can control the display unit 150 to display vital signal education data or control the output unit 160 to output vital signal education data in correspondence to the vital signal display mode.

To this end, the display unit 150 may include a display module and the output unit 160 may include a small-sized print module.

The vital signal display mode may be a mode in which output form information for displaying vital signal education data for the kind of each signal shown by vital signal recognition information has been set.

The vital signal education data may be measured from a human body in a normal healthy condition and converted for vital signal education, may be an example of predetermined vital signal data, and may be created by the processor 130.

That is, the processor 130 can control the display unit 150 or the output unit 160 to display or output vital signal education data in any one of a slope graph form, a histogram form, and a numeral form shown by the output form information.

Meanwhile, a probe attachment sensing signal and vital signal recognition information are input from the human body model 120, the processor 130 can check vital signal education information corresponding to the vital signal recognition information and can output vital signal measurement procedure information and vital signal monitoring method information included in the vital signal education information.

The vital signal measurement procedure information may be information that is used to teach a procedure of measuring a vital signal according to vital signal recognition information using the vital signal measurement probes 111, 112, and 113.

Meanwhile, the vital signal monitoring method information may be information that is used to teach a reference for determining whether the vital signal measurement data displayed on the display unit 150 are data in a normal range.

The vital signal measurement procedure information and vital signal monitoring method information may be displayed in any one of a text, an image, and a video.

Meanwhile, when an electrocardiogram education mode is entered, the processor 130 can create vital signal stop data, and can control the display unit 150 to display vital signal stop data or can control the output unit 160 to output vital signal stop data.

The vital signal stop data may be an example of vital signal data measured from a human body in a cardiac arrest condition and then converted.

When the electrocardiogram education mode is entered, the processor 130 can control expansion and contraction of the heart model 140 on the basis of vital signal stop data.

In detail, the processor can stop expansion and contraction of the heart model 140 in correspondence to vital signal stop data.

Meanwhile, the input unit 170 is connected with the cardiopulmonary resuscitation education assistant device 200 and receives cardiopulmonary resuscitation data from the cardiopulmonary resuscitation education assistant device 200, and the processor 130 can create vital signal resuscitation data on the basis of the cardiopulmonary resuscitation data and can control the display unit 150 to display the vital signal resuscitation data or can control the output unit 160 to output the vital signal resuscitation data.

The input unit 170 may be an input terminal and can connect an upper portion shape of a human body to the cardiopulmonary resuscitation education assistant device 200 through a wire Y.

The cardiopulmonary resuscitation data may be data showing the degrees of pressure and electrical stimulus that are input to the cardiopulmonary resuscitation education assistant device 200 when a learner perform cardiopulmonary resuscitation (cardiopulmonary resuscitation for an examinee to apply pressure to a heart or cardiopulmonary resuscitation that is performed by a defibrillator 220) on the cardiopulmonary resuscitation education assistant device 200.

The vital signal resuscitation data may be an example of vital signal data measured from a corresponding human body and then converted when cardiopulmonary resuscitation is performed on a human body in a cardiac arrest condition.

That is, the processor 130 can determine the degree of resuscitation of the heart and the lungs are resuscitated on the basis of the cardiopulmonary resuscitation data and can create vital signal resuscitation data in correspondence to the determined degree of resuscitation of the heart and the lungs.

The vital signal resuscitation data may show one or more of an electrocardiogram vital signal, and oxygen saturation vital signal, and a blood pressure vital signal.

Thereafter, the processor 130 can control expansion and contraction of the heart model 140 in correspondence to vital signal resuscitation data.

Meanwhile, the processor 130 can determine whether the cardiopulmonary resuscitation data satisfies a predetermined resuscitation reference, and can stop expansion and contraction of the heart model 140 when the cardiopulmonary resuscitation data does not satisfy the predetermined resuscitation reference.

The predetermined resuscitation reference may be whether the number of times of pressing the heart per unit time, the average pressure when the heart is pressed, the number of times of electrical stimulus per unit time, and the average electrical energy amount of electrical stimulus are respectively included in reference ranges.

Accordingly, it is possible to provide a learner with an example of success and fail of cardiopulmonary resuscitation.

The processor 130 can control the components of the system for experience educating vital signals and cardiopulmonary.

The processor 130 may include one or more core (not shown) and a connection channel (e.g., a bus) for transmitting/receiving a signal to/from a graphic processor (not shown) and/or another component.

The processor 130 according to an embodiment may be configured to perform the operation of the system 100 for experience educating vital signals and cardiopulmonary described above by performing one or more instructions stored in the storage 180.

Programs (one or more instructions) for processing and controlling of the processor 130 may be stored in the storage 180. The programs stored in the storage 180 may be separated into a plurality of modules in accordance with the functions of the stored programs.

Preferred embodiments of the present disclosure were described above. It would be understood by those skilled in the art that the present disclosure may be modified with departing from the scope of the present disclosure. Therefore, the disclosed embodiments should be considered in terms of explaining, not limiting. The scope of the present disclosure is not shown in the above description, but claims, and all differences within an equivalent range should be construed as being included in the present disclosure.

Although the present disclosure was described with reference to limited exemplary embodiments and drawings, the present disclosure is not limited thereto and may be changed and modified in various ways within the equivalent range of the spirit of the present disclosure and claims described below by those skilled in the art.

What is claimed is:

1. A system for experience educating vital signals and cardiopulmonary, the system comprising:
a vital signal measurement unit having a vital signal measurement probe that is attached to a learner or a human body model, measuring a vital signal from the learner through the vital signal measurement probe, converting the measured vital signal into vital signal measurement data, and outputting the vital signal measurement data together with vital signal recognition information;
a human body model formed in the shape of a human body, and outputting a probe attachment sensing signal together with vital signal identification information corresponding to a vital signal measurement region when the vital signal measurement probe is attached to the vital signal measurement region; and
a processor setting a vital signal output mode on the basis of the vital signal recognition information when the vital signal measurement data and the vital signal recognition information are input, checking output forma information corresponding to the vital signal output mode, and controlling an output unit and a display unit respectively to output and display the vital signal measurement data in correspondence to the output format information.

2. The system of claim 1, wherein the processor sets the vital signal output mode on the basis of the vital signal recognition information when the probe attachment sensing signal and the vital signal recognition information are input, checks output forma information corresponding to the vital signal output mode, and controls the output unit and the display unit respectively to output and display vital signal education data in correspondence to the output format information.

3. The system of claim 1, further comprising a heart model formed in the shape of the heart of a human body, disposed in the human body model, and expanding and contracting to represent relaxation and contraction of the heart.

4. The system of claim 1, wherein when the vital signal measurement data and the vital signal recognition information are input, the processor controls expansion and contraction of the heart model on the basis of the vital signal measurement data.

5. The system of claim 1, wherein when the probe attachment sensing signal and the vital signal recognition information are input, the processor controls expansion and contraction of the heart model on the basis of vital signal education data.

6. The system of claim 1, further comprising:
a display unit displaying the vital signal data or vital signal education data in one or more of a text, an image, and a video; and
an output unit printing out the vital signal data or the vital signal education data.
